(19) United States
(12) Patent Application Publication
(10) Pub. No.: US 2009/0192364 A1
(43) Pub. Date: Jul. 30, 2009

(54) Infant monitoring system

(76) Inventors: Andrew M. Voto, Brighton, MI (US); Ronald K. Selby, Flint, MI (US); Kenneth D. Perry, New Lothrop, MI (US); David A. Ross, Columbiaville, MI (US); Alfred V. Dumsa, JR., Brighton, MI (US)

Correspondence Address: DELPHI TECHNOLOGIES, INC., M/C 480-410-202, PO BOX 5052, TROY, MI, 48007, US

(21) Appl. No.: 12/011,651

(22) Filed: Jan. 29, 2008

(51) Int. Cl. A61B 5/00

(52) U.S. Cl. 600/301

(57) ABSTRACT

An infant monitoring system includes a bladder configured for communication with an infant having a mass and measurable biological functions and properties. The bladder contains a substantially non-toxic fluid adapted to transmit fluid pressure. The infant monitoring system also includes a single pressure sensor that selectively monitors the fluid pressure. The pressure sensor detects a presence of the infant by monitoring for a substantially continuous pressure due to the mass of the infant. The infant monitoring system also includes an electronic module operatively connected to the pressure sensor, which electronic module receives a signal indicative of the fluid pressure monitored by the pressure sensor. The electronic module emits a notification if the signal is substantially outside of a predetermined boundary.

### BACKGROUND

The present disclosure relates generally to monitors, and more particularly to a system and method for monitoring an infant.

Monitoring systems range from relatively simple systems that are capable of detecting sounds to more complex systems that are capable of detecting biological functions, such as respiration and heart rate. More complex systems may be suitable for hospital and/or home use, but generally include costly monitoring equipment. Some monitoring systems include wired electrodes that adhere to multiple locations on a person's body. In some instances, the electrodes may be uncomfortable and/or cumbersome, especially when the person is an infant. Also, a substantial amount of care for positioning the electrodes on the person's body may be necessary so that the electrodes can properly transmit electrical signals. Additionally, when using such a monitoring system, the electrodes are affixed to the person prior to monitoring, and must be removed from the person before he/she may be fully disengaged from the system. As such, using this type of monitoring system may be time-consuming and/or burdensome.

As such, it would be desirable to provide an improved method of monitoring biological functions and/or properties of an infant.

### SUMMARY

An embodiment of an infant monitoring system includes a bladder configured for communication with an infant having a mass and measurable biological functions and properties. The bladder contains a substantially non-toxic fluid adapted to transmit fluid pressure. The infant monitoring system also includes a single pressure sensor that is in operative communication with the fluid, and selectively monitors the fluid pressure. The pressure sensor detects the infant's presence by monitoring for a substantially continuous pressure due to the mass of the infant. The infant monitoring system also includes an electronic module operatively connected to the pressure sensor. The electronic module receives a signal indicative of the fluid pressure monitored by the pressure sensor. The electronic module also emits a notification if the signal is substantially outside of a predetermined boundary.

### BRIEF DESCRIPTION OF THE DRAWINGS

Features and advantages of embodiments of the present disclosure will become apparent by reference to the following detailed description and drawings, in which like reference numerals correspond to similar, though not necessarily identical components. For the sake of brevity, reference numerals or features having a previously described function may or may not be described in connection with other drawings in which they appear.

FIG. 1 is a flow diagram depicting an embodiment of a method of monitoring an infant having a mass and measurable biological functions; and

FIG. 2 is a schematic side view of an embodiment of an infant monitoring system having an infant in communication therewith.

### DETAILED DESCRIPTION

Embodiment(s) of the infant monitoring system disclosed herein advantageously utilize a single pressure sensor (as opposed to two or more pressure sensors) to monitor the presence of an infant lying in contact with a fluid-filled bladder. The system monitors one or more measurable biological functions and/or properties of the infant, and advantageously transmits a notification if the function(s) and/or properties exceed a predetermined boundary. In an embodiment, the infant monitoring system monitors the heart rate and/or respiration of an infant lying on the bladder in a contained area.

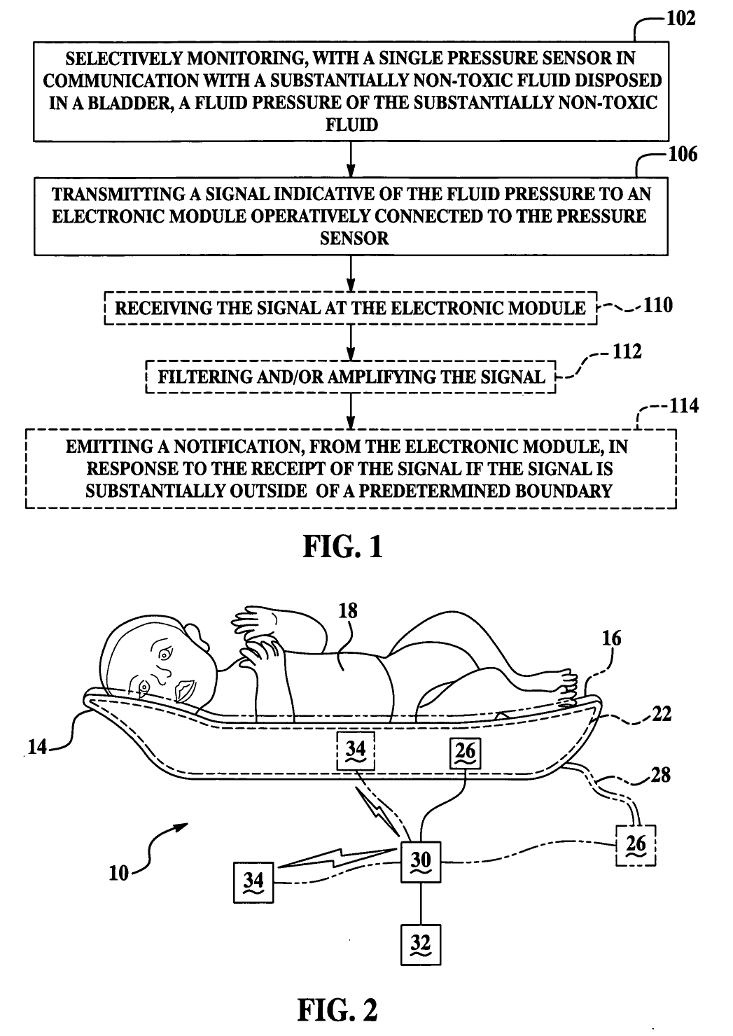

Referring now to FIG. 1, an embodiment of a method of monitoring an infant having a mass and measurable biological functions includes selectively monitoring a fluid pressure of a fluid, e.g., a substantially non-toxic fluid, disposed in a bladder, as depicted at reference numeral 102. A signal, indicative of the fluid pressure, is transmitted to an electronic module operatively connected to the pressure sensor, as depicted at reference numeral 106. The signal may be received at the electronic module, as depicted at reference numeral 110, the signal may be filtered and/or amplified, as depicted at reference numeral 112, and the electronic module emits a notification in response to the receipt of the signal if the signal is substantially outside of the predetermined boundary, as depicted at reference numeral 114. Embodiments of the system and method are discussed further hereinbelow in reference to FIG. 2.

Referring now to FIG. 2, an embodiment of the infant monitoring system 10 includes a bladder 14 configured for communication/contact with an infant 18. It is to be understood that the communication/contact between the bladder 14 and the infant 18 may be direct contact, in which nothing is positioned between the bladder 14 and the infant 18, or it may be indirect contact, in which something (e.g., clothing worn by an infant 18, bedding (e.g., material layer 16 discussed further hereinbelow) disposed on bladder 14, and/or the like) is positioned between the bladder 14 and the infant 18.

In a non-limiting embodiment, the bladder 14 is capable of receiving an infant 18 weighing from about four pounds to about twenty-five pounds.

The bladder 14 contains a fluid 22 configured to transmit fluid pressure. In an embodiment, the fluid 22 is a substantially non-toxic fluid. As non-limiting examples, the fluid 22 may be selected from air and/or nitrogen, or fluid 22 may be selected from a substantially non-toxic, minimally compressible or non-compressible fluid selected from glycerine, silicone, water, and/or combinations thereof. It is to be understood that the bladder 14 is formed from a material that is substantially impermeable to the fluid 22. The bladder 14 may also be formed from a substantially flexible material. Non-limiting examples of such materials include rubber, coated fabrics, and/or combinations thereof. The bladder 14 material may also advantageously be compatible with the fluid 22. Some non-limiting examples of alternate bladder 14 materials include elastomers and barrier polymeric materials such as, for example, latex, EPDM (ethylene propylene diene monomer), EVOH (ethylene vinyl alcohol copolymer), nitrile rubber, PVC (polyvinyl chloride), polyesters, or the like, or combinations thereof.

The bladder 14, or portions thereof, may be configured to substantially reduce fluid 22 leakage if the bladder 14 is punctured. As a non-limiting example, the bladder 14 may be formed, at least partially, from a substantially self-healing material in a suitable design/configuration and wall thickness. Any suitable self-healing material may be used, e.g., self-healing materials used for automobile tires.

In an embodiment, the bladder 14 includes a material layer 16 which covers at least a portion of the bladder 14. In one embodiment, the portion of the bladder 14 covered by the material layer 16 is an area that may contact the infant 18. It is to be understood that the material layer 16 may provide a contact area for the infant 18 that is substantially warmer, cooler, softer, smoother, and/or plusher than the bladder 14, as desired. The material layer 16 may also provide insulating properties. The material layer 16 generally conforms to the shape of the bladder 14, and as such, is formed of a substantially flexible material, examples of which include cotton fabrics, polyester fabrics, or the like, or combinations thereof. The material layer 16 may be removable from, partially removable from, or permanently fixed to the bladder 14. As an example, the material layer 16 may be a sleeve that receives at least a portion of the bladder 14.

When in communication with the bladder 14, an infant 18 may, for example, sit/recline against or lie on the bladder 14. As such, at least a portion of the infant's 18 abdomen and/or thorax is in communication with the bladder 14. In an embodiment, the bladder 14 is configured for use in a contained area such as, for example, cribs, bassinettes, playpens, highchairs, strollers, infant carriers, infant car seats, or the like, or combinations thereof.

A single pressure sensor 26 is in operative communication with the fluid 22. It is to be understood that the single pressure sensor 26 selectively monitors the pressure of the fluid 22. “Selective monitoring,” as used herein, means that monitoring is accomplished as desired and/or required, and that monitoring may be paused or stopped as desired and/or required. As such, the pressure of the fluid 22 may be monitored substantially continuously while the system 10 is operable, or may be monitored for some duration while the system 10 is operable. As a non-limiting example, the pressure of the fluid 22 may be monitored during the entire time period that the system 10 is powered on, unless the system 10 is manually paused, such as by a user. As another non-limiting example, the pressure of the fluid 22 may be briefly monitored (i.e., for about 0.1 second, about 1 second, or about 5 seconds) at substantially regular predetermined intervals, which may be about five seconds, about ten seconds, or any desirable length of time. In other embodiments, the predetermined monitoring intervals may be of a longer or shorter duration and/or may be irregular in duration, as desired.

It is to be understood that the sensor 26 may be in direct or indirect communication with the fluid 22. In one embodiment, the sensor 26 is positioned within the bladder 14 in direct contact with the fluid 22. In another embodiment, the sensor 26 is positioned within the bladder 14 and in communication with the fluid 22, but has a protective barrier established thereon. In still another embodiment, the sensor 26 is positioned outside the bladder 14, but in communication with fluid 22. In the latter embodiment, the pressure sensor 26 may be in communication with the fluid 22 via, for example, a fluid conduit 28. When used, it is to be understood that the fluid conduit 28 is configured to substantially reduce the risk of self-entanglement and/or entanglement of the conduit 28 with another object. As a non-limiting example, the fluid conduit 28 may be formed as a substantially flat or thin ribbon or tube.

As used herein, a “single pressure sensor” 26 means one pressure sensor 26, without the inclusion of additional pressure sensors 26. The pressure sensor 26 detects the presence of the infant 18 (i.e., the infant 18 is in contact with the bladder 14) by monitoring the fluid 22 pressure. It is to be understood that the fluid pressure may change in response to the mass of the infant 18 in contact with the bladder 14 and/or in response to the infant's 18 measurable biological functions/properties. More specifically, the mass of the infant 18 lying on the bladder 14 causes the bladder 14 to compress, thereby increasing the pressure of the fluid 22 located therein. Similarly, a force may be exerted on the bladder 14 when an infant's 18 body physically responds to his/her heart beat and/or respiration. Such forces also cause the bladder 14 to compress, which changes the pressure of the fluid 22.

In an embodiment, the presence of the infant 18 is detected when the pressure sensor 26 detects a substantially continuous pressure in the fluid 22, accompanied by intervals of small pressure changes. The substantially continuous pressure is due to the mass of the infant 18 in communication with the bladder 14. The system 10 is generally configured to monitor a base pressure of the fluid 22, and when the infant 18 is placed in contact with the bladder 14, the base pressure changes as a result of the applied mass.

The smaller changes in fluid pressure may result from at least one of the infant's 18 measurable biological functions and/or properties. Non-limiting examples of a biological function include respiration intensity, respiration rate, heart rate, and/or combinations thereof. Many of the infant's 18 biological functions result in very little external body movement, which impose a relatively small change on the fluid pressure in the bladder 14. It is to be understood that the pressure sensor 26 is configured with sufficient sensitivity to detect such changes in fluid pressure. In some instances, detection of the changes in fluid pressure may render more useful results when the infant 18 is relatively still, as opposed to when the infant 18 is engaging in substantial gross motor activity. It is to be further understood that the pressure sensor 26 may monitor more than one measurable biological function and/or property substantially simultaneously.

The pressure sensor 26 may recognize the initial pressure change (due to the compression of the bladder 14 by the infant's 18 mass) and the subsequent smaller pressure changes (due to the biological functions), and the system 10 equates such changes with the presence of the infant 18. Sensor 26 recognizing the initial pressure change (e.g., when the infant 18 is laid down), may be advantageous for automatically exiting a low power mode (discussed further below).

However, the single pressure sensor 26 of the present disclosure does not require a change in pressure (e.g., when the infant 18 is initially laid down) in order to detect the presence of the infant 18. This may be advantageous if there is a malfunction or glitch in operation of the system 10, and the pressure sensor 26 wakes up “dumb” while the infant 18 is already lying on the bladder 14. In this instance, the pressure sensor 26 is able to detect the presence of the infant 18 without having to wait for the infant 18 to be picked up and laid down again on the bladder 14.

An electronic module 30 is operatively connected to the pressure sensor 26. The electronic module 30 receives signal(s) from the pressure sensor 26, which are indicative of the fluid pressure monitored by the pressure sensor 26.

In an embodiment, the electronic module 30 includes suitable electronic circuitry to perform a desired task. The electronic module 30 may be mounted on the interior or exterior of the bladder 14. It is to be understood that the electronics module may be releasably, semi-releasably, or permanently engaged or attached to the bladder 14. In a non-limiting example, the electronic module 30 is mounted in a pocket (not shown) formed in a wall of the bladder 14.

The electronic module 30 may receive the signal(s) in any suitable form. As non-limiting examples, the signal may correspond to the substantially instantaneous pressure values, as detected by the pressure sensor 26 during monitoring, and/or the signal may include data representative of variations in the values detected by the pressure sensor 26 during monitoring.

The pressure sensor 26, in combination with the electronic module 30, may utilize one or more algorithms and/or filters to monitor the initial change in fluid pressure and the minor variations in the fluid pressure. A non-limitative example of a minor variation in fluid pressure is low amplitude sonic vibration. Additionally, the pressure sensor 26 and electronic module 30 may amplify, isolate or filter out at least a portion of a particular signal. In one non-limiting example, relatively large amplitude fluid pressure changes may be indicative of infant 18 gross motor activity. In some instances, it may be desirable to filter such changes from the transmitted signals. In another non-limiting example, a portion of the signal that is indicative of a biological function/property may be amplified. It is to be understood that the signal may be amplified, isolated or filtered by any suitable method(s) to achieve a desirable result. For example, if desired, a band pass filter may be used to separate the respiration pressure signal from the composite pressure signal. In an embodiment, the body temperature of the infant 18 may also be monitored by including a thermocouple or thermistor (not shown) in communication with the bladder 14.

The electronic module 30 may then derive the respiration rate and/or intensity, heart rate, body temperature, and/or combinations thereof from the received signals.

The electronic module 30 is programmed with predetermined settings for each biological function and/or property. The predetermined settings may be particular to the infant 18, and may be based on for example, the infant's weight, normal heart rate, normal respiration rate, and/or normal respiration intensity. The predetermined settings may be a defined range (having lower and upper acceptable limits to define a predetermined boundary) of suitable values for the detected biological functions and/or properties. In a non-limiting example, the predetermined settings include an average infant heart rate ranging from about 100 beats/minute to about 150 beats/minute, an average infant respiration rate ranging from about 30 breaths/minute to about 60 breaths/minute, and a suitable respiration intensity. As defined herein, a “suitable respiration intensity” refers to the ability to adequately ensure appropriate oxygenation for an infant population.

Upon receiving the signals from the pressure sensor 26, the electronic module 30 determines the detected heart rate, respiration rate, and/or respiration intensity. If the detected rates are substantially outside of a predetermined boundary (e.g., by falling below or exceeding the predetermined settings) programmed in the module 30, the electronic module sends a notification signal to a notification unit 34.

Upon receiving the notification, the notification unit 34 may respond by emitting an alarm. As non-limiting examples, the notification may be transmitted to the notification unit 34 via a wire, radio frequency (RF), satellite, telecommunications (e.g., phones, text messages, emails, etc.), optical communications (e.g., infrared signals), and/or the like, and/or combinations thereof. In an embodiment, the notification, transmitted via radio frequency, uses the Zigbee standard. It is to be understood that the Zigbee standard may advantageously enable reduced power consumption by the system 10.

The alarm emitted by the notification unit 34 may be, for example, an audible alarm, a visual alarm, a tactile alarm, and/or combinations thereof. In an embodiment, the audible alarm includes a pre-recorded message and/or a series of sounds (e.g., “beeps”). In another embodiment, the alarm includes one or more pre-recorded messages that verbally indicate the nature of the alarm. As an example, the notification unit 34 may emit an alarm, which announces, “Heart rate below X” (beats per minute) and/or “Respiration rate below Y” (breaths per minute). In yet another embodiment, the alarm is embodied as a sound or series of sounds that indicates the nature of the alarm. As non-limiting examples, the alarm may be a series of short beeps (e.g., indicating a high heart rate), and/or a sequence of one short beep and one long beep (e.g., indicating a high respiration rate).

A non-limiting example of a visual alarm is a light that turns on, flashes and/or changes color upon receiving the notification. A tactile alarm may cause a vibration, which may be detected by a user/caretaker. It is to be understood that a tactile alarm may be desirable, for example, for a hearing impaired and/or visually impaired caretaker, and/or may be desirable to preserve a quiet environment. As a non-limiting example, a tactile alarm may vibrate a receiver, located on or in an object associated with a caretaker (e.g., a wristwatch). In an embodiment, a tactile alarm is utilized in conjunction with an audible alarm and/or a visual alarm.

The alarm and the nature of the notification associated with the alarm may be default settings (e.g., set by the manufacturer) and/or may be user-defined.

It is to be understood that the notification unit 34 may be located in any suitable location, as desired. As such, the notification unit 34 may be located proximate to the single pressure sensor 26, remote from the single pressure sensor 26, or combinations thereof. For example, the notification unit 34 may be located close to a caregiver (remote from the infant 18), or the notification unit 34 may be located close to the infant 18, or the notification unit 34 may be close to the caregiver, and a second notification unit may be close to the infant 18.

In an embodiment, the monitoring system 10 may trigger a remote and/or local audible and/or visual alarm when the infant 18 is in some form of distress. In another embodiment, the system 10 may further provide a local sonic/audible and/or tactile/vibrational stimulus to the infant 18.

The electronic module 30 may receive power from a power source 32 such as, for example, a battery and/or an AC/DC transformer. As a non-limiting example, the power source 32 may include a piezoelectric battery. The power source 32 may be replaceable and/or rechargeable. In an embodiment, the power source 32 is recharged via inductive charging.

It is to be understood that the power source 32 may be mounted at least partially within the bladder 14, such as, for example, within a pocket formed substantially in a wall of the bladder 14. It is to be further understood that appropriate levels of electrical isolation may be desirable when mounting certain power sources 32 (e.g., a transformer) within or on the bladder 14.

The infant monitoring system 10 may operate in a low power mode when an infant 18 is not detected in communication with the bladder 14. In an embodiment, during a low power mode, the pressure sensor 26 monitors the pressure of the fluid 22 for relatively short predetermined monitoring periods. These monitoring periods are followed by longer intervals at which no monitoring is accomplished. As a non-limiting example, when the system 10 is in low power mode, the pressure of the fluid 22 is briefly monitored (e.g., for about 0.1 second, about 1 second, or about 5 seconds), and then the system 10 is inoperable for a predetermined interval (e.g., about one minute, about five minutes, about ten minutes, etc.) until the next monitoring period takes place.

In an embodiment, the alarm (discussed above) may be programmed to be triggered in the event that the infant 18 is removed from the bladder 14. This may, in some instances, potentially assist in substantially preventing unauthorized personnel from removing the infant 18 from the bladder 14.

It is to be understood that the combination of the relatively constant fluid pressure (due to the infant's mass) and the relatively small changes in fluid pressure (due to the biological functions) indicate to the system 10 that the infant 18 is in communication therewith. In an embodiment, if a mass is detected without biological function(s), the electronic module 30 will emit a notification.

While several embodiments have been described in detail, it will be apparent to those skilled in the art that the disclosed embodiments may be modified. Therefore, the foregoing description is to be considered exemplary rather than limiting.

What is claimed is:

1. An infant monitoring system, comprising:
a bladder configured for communication with an infant having a mass and measurable biological functions and properties, the bladder containing a fluid adapted to transmit fluid pressure;
a single pressure sensor in operative communication with the fluid and configured to selectively monitor the fluid pressure, the pressure sensor configured to at least detect a presence of the infant by monitoring for a substantially continuous pressure due to the mass of the infant; and
an electronic module operatively connected to the pressure sensor and configured to receive a signal indicative of the fluid pressure monitored by the pressure sensor, wherein the electronic module is configured to emit a notification if the signal is substantially outside of a predetermined boundary.

2. The infant monitoring system of claim 1 wherein the fluid is selected from air and nitrogen, or is selected from a substantially non-toxic, minimally compressible or non-compressible fluid selected from silicone, water, glycerine, and combinations thereof.

3. The infant monitoring system of claim 1 wherein the signal indicative of the fluid pressure is also indicative of at least one of the measurable biological functions.

4. The infant monitoring system of claim 3 wherein the at least one of the measurable biological functions is selected from respiration intensity, respiration rate, heart rate, body temperature, and combinations thereof.

5. The infant monitoring system of claim 1, further comprising a notification unit configured to receive the notification and emit an alarm in response to receipt of the notification.

6. The infant monitoring system of claim 5 wherein the notification is transmitted to the notification unit via a wire, radio frequency, satellite, telecommunications, optical communications, or combinations thereof.

7. The infant monitoring system of claim 5 wherein the alarm is selected from an audible alarm, a visual alarm, a tactile alarm, and combinations thereof.

8. The infant monitoring system of claim 7 wherein the notification unit is located proximate to the single pressure sensor, remote from the single pressure sensor, or combinations thereof.

9. The infant monitoring system of claim 1, further comprising a material layer covering at least a portion of the bladder.

10. The infant monitoring system of claim 1 wherein the infant monitoring system is configured to operate in a low power mode when the infant is not in communication with the bladder.

11. The infant monitoring system of claim 1 wherein the electronic module is housed at least partially within the bladder.

12. The infant monitoring system of claim 1 wherein the predetermined boundary is selected from a heartbeat range, a respiration rate range, a respiration intensity range, and combinations thereof.

13. A method of monitoring an infant having a mass and measurable biological functions, the method comprising:
selectively monitoring, with a single pressure sensor in communication with a fluid disposed in a bladder, a fluid pressure of the fluid, the bladder configured for communication with the infant, the single pressure sensor configured to at least detect a presence of the infant by monitoring the fluid pressure for a substantially continuous pressure due to the mass of the infant; and
transmitting a signal indicative of the fluid pressure to an electronic module operatively connected to the pressure sensor, wherein the electronic module is configured to emit a notification if the signal is substantially outside of a predetermined boundary.

14. The method of claim 13 wherein the fluid is selected from air and nitrogen, or is selected from a substantially non-toxic, minimally compressible or non-compressible fluid selected from silicone, water, glycerine, and combinations thereof; and wherein the signal indicative of the fluid pressure is also indicative of at least one of the measurable biological functions.

15. The method of claim 14 wherein the at least one of the measurable biological functions is selected from respiration intensity, respiration rate, heart rate, body temperature, and combinations thereof.

16. The method of claim 13, further comprising:
receiving the notification at a notification unit; and
emitting an alarm in response to receipt of the notification.

17. The method of claim 16 wherein the notification is transmitted to the notification unit via a wire, radio frequency, satellite, telecommunications, optical communications, and combinations thereof; and wherein the alarm is emitted audibly, visually, tactilely, or combinations thereof.

18. The method of claim 13, further comprising operating in a low power mode after detecting a lack of the presence of the infant.

19. The method of claim 13, further comprising triggering an alarm when the infant is removed from the bladder.

20. The method of claim 13 wherein the predetermined boundary is selected from a heartbeat range, a respiration rate range, a respiration intensity range, and combinations thereof.

21. A method of making an infant monitoring system, the method comprising:
providing a bladder containing a fluid adapted to transmit fluid pressure, the bladder configured for communication with an infant having a mass and measurable biological functions and properties;
positioning a single pressure sensor in operative communication with the fluid, the pressure sensor configured to selectively monitor the fluid pressure and to at least detect a presence of the infant by monitoring for a substantially continuous pressure due to the mass of the infant; and
operatively connecting an electronic module to the pressure sensor, the electronic module configured to receive a signal indicative of the fluid pressure monitored by the pressure sensor, wherein the electronic module is configured to emit a notification if the signal is substantially outside of a predetermined boundary.
